(19) United States
(12) Patent Application Publication
(10) Pub. No.: US 2021/0118543 A1
(43) Pub. Date: Apr. 22, 2021

(54) Robotic Systems and Related Methods for Dispensing a Substance

(71) Applicant: Cellink AB, Göteborg (SE)

(72) Inventors: Erik Gatenholm, Göteborg (SE); Hector Martinez, Göteborg (SE)

(21) Appl. No.: 16/965,333

(22) PCT Filed: Feb. 1, 2019
(86) PCT No.: PCT/EP2019/052480

§ 371 Date: Jul. 28, 2020

Related U.S. Application Data
(60) Provisional application No. 62/625,596, filed on Feb. 2, 2018.

(30) Foreign Application Priority Data
Mar. 26, 2018 (SE) 1850334-2

(51) Int. Cl. G16H 20/17; G06N 20/00; G05B 19/4155

(52) U.S. Cl. CPC G16H 20/17; G05B 2219/50391; G05B 19/4155; G06N 20/00

(57) ABSTRACT

The present disclosure relates to a a computer implemented method for determining a substance dispensing plan. The method comprises obtaining (S10) a digital representation of a set of a person's current bodily features. The method further comprises determining (S20) a substance dispensing plan based on a comparison between the digital representation of the set of the person's current bodily features and a set of desired bodily features. The present disclosure also relates to corresponding computer programs, control systems and robotic systems.

### TECHNICAL FIELD

The present disclosure relates to methods and systems for planning a procedure and associated computer programs.

### BACKGROUND

The need for surgery comprising steps acting in subcutaneous regions of a patient is constantly rising. The need for introducing a substance into a subcutaneous region of a patient is typically needed as part of such surgical procedures. For instance, in cosmetic surgery filler may be introduced into a subcutaneous region in order to smooth out a wrinkle.

The substances are often delivered transcutaneously, which may be associated with a number of potential problems. For instance, hematoma, nerve damage, infection and scarring may constitute potential problems.

Another problem associated primarily with cosmetic surgery is the risk of the surgical outcome not matching the desired outcome. The patient is typically at the mercy of the surgeon, who has all the problems associated with being human, such as problems associated with concentration, precision in motor skills and the need to keep skills current through constant practice.

There is thus a need in the art for methods and systems which is able to provide improved aesthetical outcomes and at the same time reduce the risk of complications.

### SUMMARY

The present invention draws on the strengths of image processing in many different applications, in particular in combination with artificial intelligence, to determine how to optimally administer a substance, such as a filler, medicine, anaesthesia and/or a vaccine, into a subcutaneous region. The present disclosure further relates to systems for carrying out the determined plan of how to optimally administer the substance.

In particular, the present disclosure relates to a computer implemented method for determining a substance dispensing plan. Such method may be used for e.g. transcutaneous delivery of a substance into a subcutaneous region. The method comprises obtaining a digital representation of a set of a person's current bodily features. The method further comprises determining a substance dispensing plan based on a comparison between the digital representation of the set of the person's current bodily features and a set of desired bodily features. The method thereby enables automatic methods for determining how to optimally dispense the substance into the subcutaneous region. The method further enables the integration of artificial intelligence methods for determining the substance dispensing plan, which may provide a more accurate substance dispensing plan than a human counterpart would be able to do. In other words, the method is thereby able to provide plans for dispensing a substance into a subcutaneous region faster and more accurate than a human counterpart would be able to do. The improved accuracy of the substance dispensing plan translates to reduced treatment times and/or a reduced probability for complications, while simultaneously achieving superior results compared to the technology of the prior art.

According to some aspects, the method further comprises comparing the digital representation of the set of the person's current bodily features to a digital representation of a set of desired bodily features. Comparing the digital representation to the set of desired bodily features enables determining the differences between the digital representation of the set of the person's current bodily features and the digital representation of a set of desired bodily features. The comparison can thereby produce input, in particular in the form of the differences between current and desired bodily features, for the determination of the substance dispensing plan.

According to some aspects, obtaining a digital representation of a set of a person's current bodily features further comprises scanning the person's face, and generating a digital representation of a set of the person's current facial features. Scanning the person's face is a quick and accurate way to obtain a digital representation of a set of the person's current facial features. Scanning the person's face further enables obtaining a digital representation from different angles and at different distances, thereby enabling obtaining an accurate, three-dimensional digital representation of the set of the person's current facial features.

According to some aspects, the step of generating a digital representation of a set of the person's current facial features is performed, at least in part, using facial recognition. Facial recognition can efficiently identify a set of facial features, and can provide an efficient digital representation of the set of the person's current facial features. Facial recognition may further be used in combination with artificial intelligence in order to efficiently determine the substance dispensing plan.

According to some aspects, the method further comprises comparing the digital representation of the set of the person's current facial features to a set of desired facial features. Comparing the digital representation to the set of desired facial features enables determining the differences between the digital representation of the set of the person's current facial features and the digital representation of a set of desired facial features. The comparison can thereby produce input, in particular in the form of the differences between current and desired facial features, for the determination of the substance dispensing plan.

According to some aspects, the method further comprises determining the presence of one or more wrinkles based on the obtained digital representation of the set of the person's current bodily features. According to some aspects, the method further comprises determining how much substance to place under the skin of the person to fill the one or more wrinkles based on the comparison between the digital representation of the set of the person's current bodily features and the set of desired bodily features. A particular strength of the disclosed method is its ability to enable to both identify and cosmetically remedy wrinkles.

According to some aspects, determining the substance dispensing plan comprises using, at least in part, an artificial intelligence algorithm for making the comparison between the digital representation of the set of the person's current bodily features and the set of desired bodily features. The use of an artificial intelligence algorithm can increase both the accuracy of the substance dispensing plan and the determination of what are the relevant steps and parameters of the plan. The artificial intelligence algorithm may also reduce treatment times and probability of complications associated with dispensing the substance.

According to some aspects, the substance dispensing plan comprises at least one of a syringe skin penetration location, a syringe skin penetration attitude, and a syringe skin penetration depth.

According to some aspects, the method may further comprise dispensing the substance, via a syringe, based on the determined substance dispensing plan. Syringes provide straightforward ways of transcutaneous delivery of most substances. Syringes may be reused several times during treatment. Syringes further offer the advantage of easily administer the substance at different locations and/or attitudes.

According to some aspects, the substance dispensing plan comprises dispensing substance at a substance dispensing volume at a subcutaneous region of the person. The inclusion of a syringe skin penetration location, a syringe skin penetration attitude, a syringe skin penetration depth and/or dispensing substance at a substance dispensing volume at a subcutaneous region of the person thereby enables carrying out the method manually, semi-automatically or fully automatically.

According to some aspects, determining the substance dispensing plan comprises determining at least one instruction for control of movement of a robotic arm holding the syringe based on the determined syringe skin penetration location(s) and/or syringe skin penetration attitude(s) and/or syringe skin penetration depth(s). The determination of the substance determining plan may comprise determining at least one instruction for control of dispensing of the substance via the syringe, said at least one instruction for dispensing of the substance of the syringe being correlated to the instructions for control of movement of the robotic arm.

The present disclosure further relates to a computer program comprising computer program code which, when executed, causes a robotic system to carry out the method as described above and below. The computer program is configured to carry out the disclosed method for determining a substance dispensing plan and therefore has all the associated technical effects and advantages.

The present disclosure also relates to a control system for determining a substance dispensing plan for e.g. transcutaneous delivery of substance into a subcutaneous region. The control system may also control, fully or in part, the following transcutaneous delivery of substance into a subcutaneous region. The control system comprises control circuitry. The control circuitry is configured to carry out the method as described above and below. The control system is configured to cause a system which it controls to carry out the disclosed method for determining a substance dispensing plan, and optionally executing transcutaneous delivery of substance into a subcutaneous region and therefore has all the associated technical effects and advantages. The control system can be integrated into a system having components necessary to carry out the disclosed method, thereby extending the functionality of existing systems or integrating separate systems into a single, larger system with extended functionality.

According to some aspects, the control circuitry comprises a processor and a memory, wherein the memory is configured to store a computer program as described above and below thereon, and wherein the processor is configured to execute the computer program stored on the memory.

The present disclosure further relates to a robotic system for determining and executing a substance dispensing plan. The robotic system comprises a robotic arm. The robotic system further comprises a substance delivery system configured to dispense the substance into a subcutaneous region. The robotic system also comprises at least one camera. The robotic system additionally comprises control circuitry. The at least one camera is configured to obtain a digital representation of a set of a person's current bodily features. The control circuitry is configured to determine a substance dispensing plan based on a comparison between the digital representation of the set of the person's current bodily features and a set of desired bodily features. The robotic system physically implements a system able to carry out the method for determining and executing a substance dispensing plan, for e.g. transcutaneous delivery of a substance into a subcutaneous region, and thus has all the technical effects and advantages of the disclosed methods.

According to some aspects, the substance delivery system is configured to dispense the substance based on the determined substance dispensing plan. The robotic system is thereby further configured to carry out the determined substance dispensing plan.

According to some aspects, the control circuitry comprises a processor and a memory. The memory is configured to store a computer program as described above and below thereon. The processor is configured to execute the computer program stored on the memory.

According to some aspects, the substance delivery system comprises a syringe arranged at the robotic arm. The substance delivery system is configured to dispense the substance via the syringe. The ability for transcutaneous delivery of the substance is thereby integrated into the substance delivery system.

According to some aspects, the substance delivery system is configured to receive a cartridge comprising a syringe. The cartridge comprises the substance. The substance delivery system is configured to dispense the substance via the syringe. The ability for transcutaneous delivery of the substance is thereby provided by the received cartridge, and the substance delivery system is configured to deliver the substance indirectly by acting on the cartridge to cause the substance to be dispensed via the syringe.

According to some aspects, the robotic arm is configured to move in six degrees of freedom. The ability to move in six degrees of freedom greatly extends the range of possible treatments as well as the degree to which a desired result can be achieved. In particular, a six degree of freedom robotic arm is able to perform a transcutaneous penetration at a wide range of attitudes.

According to some aspects, the control circuitry is further configured to compare the digital representation of the set of the person's current bodily features to a digital representation of a set of desired bodily features. Comparing the digital representation to the set of desired bodily features enables determining the differences between the digital representation of the set of the person's current bodily features and the digital representation of a set of desired bodily features. The comparison can thereby produce input, in particular in the form of the differences between current and desired bodily features, for the determination of the substance dispensing plan.

According to some aspects, the at least one camera is configured to scan the person's face. The control circuitry is configured to generate a digital representation of a set of the person's current facial features based on the scan. Scanning the person's face is a quick and accurate way to obtain a digital representation of a set of the person's current facial features. Scanning the person's face further enables obtaining a digital representation from different angles and at different distances, thereby enabling obtaining an accurate, three-dimensional digital representation of the set of the person's current facial features.

According to some aspects, the control circuitry is configured to generate the digital representation of the set of the person's current facial features, at least in part, using facial recognition. Facial recognition can efficiently identify a set of facial features, and can provide an efficient digital representation of the set of the person's current facial features. Facial recognition may further be used in combination with artificial intelligence in order to efficiently determine the substance dispensing plan.

According to some aspects, the comparison between the digital representation of the set of the person's current bodily features and the set of desired bodily features comprises a comparison between the digital representation of the set of the person's current facial features and a set of desired facial features. Comparing the digital representation to the set of desired facial features enables determining the differences between the digital representation of the set of the person's current facial features and the digital representation of a set of desired facial features. The comparison can thereby produce input, in particular in the form of the differences between current and desired facial features, for the determination of the substance dispensing plan.

According to some aspects, the control circuitry is configured to determine the substance dispensing plan using, at least in part, an artificial intelligence algorithm for making the comparison between the digital representation of the set of the person's current bodily features and the set of desired bodily features. The use of an artificial intelligence algorithm can increase both the accuracy of the substance dispensing plan and the determination of what are the relevant steps and parameters of the plan. The artificial intelligence algorithm may also reduce treatment times and probability of complications associated with dispensing the substance.

According to some aspects, the substance dispensing plan comprises at least one of a syringe skin penetration location, a syringe skin penetration attitude, and a syringe skin penetration depth.

According to some aspects, the substance dispensing plan comprises dispensing substance at a substance dispensing volume at a subcutaneous region of the person. The inclusion of a syringe skin penetration location, a syringe skin penetration attitude, a syringe skin penetration depth and/or dispensing substance at a substance dispensing volume at a subcutaneous region of the person thereby enables carrying out the method manually, semi-automatically or fully automatically.

According to some aspects, determining the substance dispensing plan comprises determining at least one instruction for control of movement of a robotic arm holding the syringe based on the determined syringe skin penetration location(s) and/or syringe skin penetration attitude(s) and/or syringe skin penetration depth(s).

According to some aspects, determining the substance determining plan comprises determining at least one instruction for dispensing of the substance via the syringe, said at least one instruction for dispensing of the substance of the syringe being correlated to the instructions for control of movement of the robotic arm.

According to some aspects, the control circuitry is further configured to determine the presence of one or more wrinkles based on the obtained digital representation of the set of the person's current bodily features. According to some aspects, the control circuitry is further configured to determine how much substance to place under the skin of the person to fill the one or more wrinkles based on the comparison between the digital representation of the set of the person's current bodily features and the set of desired bodily features. A particular strength of the disclosed method is its ability to enable to both identify and cosmetically remedy wrinkles.

According to some aspects, the substance delivery system is configured to dispense hyaluronic acid via the syringe. The robotic system is thereby configured to provide a dermal filler for cosmetic surgery, such as smoothing wrinkles.

### BRIEF DESCRIPTION OF THE DRAWINGS

FIG. 1 illustrates method steps of a method for determining a substance dispensing plan;

FIG. 2 illustrates a control system for for determining a substance dispensing plan; and

FIGS. 3 and 3b illustrate robotic systems for determining and executing a substance dispensing plan.

### DETAILED DESCRIPTION

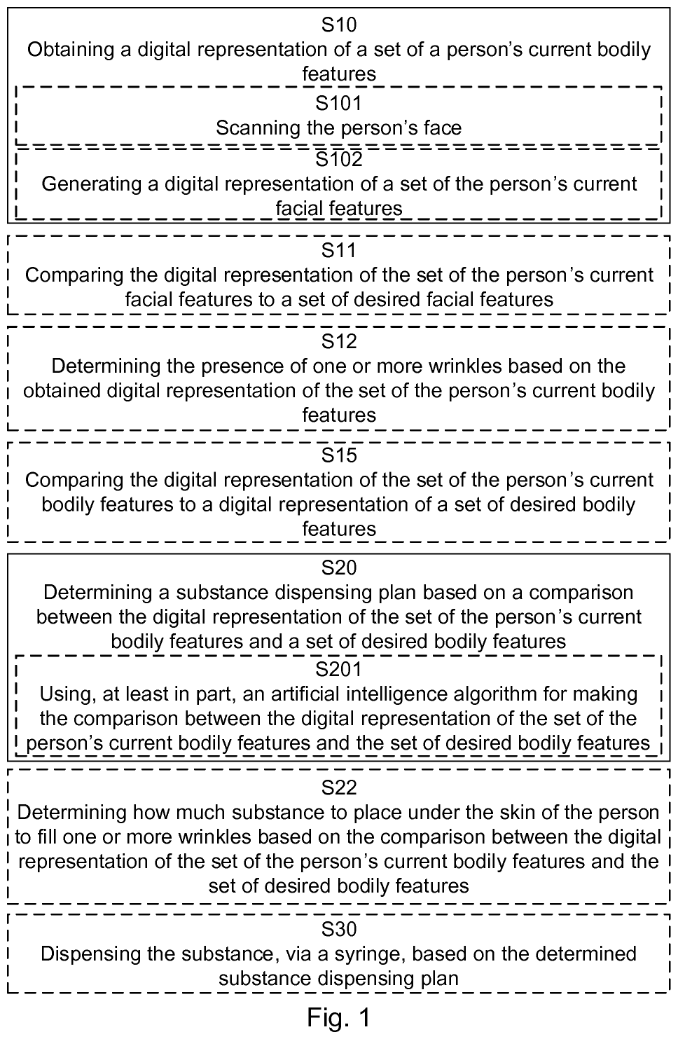

FIG. 1 illustrates method steps of a method for determining a substance dispensing plan and optionally execution such a method.

The basic idea of the disclosed method is to use the digital representation to obtain information relating to a current state of the person, and to compare the current state with a desired state in order to determine the most effective way to get from the current state to being as close to the desired state as possible.

Thus, the method comprises obtaining S10 a digital representation of a set of a person's current bodily features.

While the digital representation of the set of the person's current bodily features will be described herein as mainly relating to static features, such as facial features, it is to be understood that the current bodily features may relate to dynamic features as well, such as pupil dilation and/or movement of the chest during breathing. The digital representation may also include information relating to things happening within the person, e.g. heart rate, blood pressure and/or blood flow, e.g. as seen with an infra-red camera. According to some aspects, the digital representation comprises information relating to mechanical properties of the subcutaneous region, such as lumps, bumps, cysts and/or swellings occurring under the skin of the person.

The method further comprises determining S20 a substance dispensing plan based on a comparison between the digital representation of the set of the person's current bodily features and a set of desired bodily features.

The method thereby enables automatic methods for determining how to optimally dispense the substance into the subcutaneous region. The method further enables the integration of artificial intelligence methods for determining the substance dispensing plan, which may provide a more accurate substance dispensing plan than a human counterpart would be able to do. In other words, the method is thereby able to provide plans for dispensing a substance into a subcutaneous region faster and more accurate than a human counterpart would be able to do. The improved accuracy of the substance dispensing plan translates to reduced treatment times and/or a reduced probability for complications, while simultaneously achieving superior results compared to the technology of the prior art.

In order to facilitate the determination of the substance dispensing plan, the method preferably comprises comparing S15 the digital representation of the set of the person's current bodily features to a digital representation of a set of desired bodily features. A comparison typically helps identifying regions on the surface of the person differing sufficiently from the set of desired bodily features. A great advantage of the comparison is that, in combination with an artificial intelligence algorithm, the act of comparing S15 may be used to train the artificial intelligence algorithm to identify criteria for determining S20 the substance dispensing plan.

According to some aspects, the substance dispensing plan comprises at least one of a syringe skin penetration location, a syringe skin penetration attitude, and a syringe skin penetration depth. According to some aspects, the substance dispensing plan comprises dispensing substance at a substance dispensing volume at a subcutaneous region of the person. These are factors that an artificial intelligence algorithm is particularly suitable for determining. Since artificial intelligence algorithms, e.g. in the field of machine learning, herein considered to be a subfield of artificial intelligence, may be trained to perform many tasks at or above human-level performance, the disclosed method may be automatized to operate in a semi-automatic or automatic manner with results at or above human-level performance. Not only may procedures, e.g. in the field of cosmetic surgery, result in a more aesthetically pleasing outcome, but may also save time and/or reduce the risk of complications when implemented according to the determined substance dispensing plan.

Thus, according to some aspects, determining S20 the substance dispensing plan comprises using S201, at least in part, an artificial intelligence algorithm for making the comparison between the digital representation of the set of the person's current bodily features and the set of desired bodily features.

The method is particularly suitable for determining a substance dispensing plan for skin treatment related cosmetic surgery procedures, such as getting rid of wrinkles.

According to some aspects, obtaining S10 a digital representation of a set of a person's current bodily features further comprises scanning S101 the person's face, and generating S102 a digital representation of a set of the person's current facial features. Scanning the person's face enables obtaining a digital representation from different angles and at different distances, thereby enabling obtaining an accurate, three-dimensional digital representation of the set of the person's current facial features. Scanning the person's face is a quick and accurate way to obtain a digital representation of a set of the person's current facial features.

According to some aspects, the step of generating S102 a digital representation of a set of the person's current facial features is performed, at least in part, using facial recognition. Facial recognition algorithms enable identifying distinguishing features and/or performing statistical analysis of the scan of the person's face in order to distill the scan into values and comparing the values with templates to eliminate variances. The facial recognition may be used in combination with 3D sensors in order to capture information about the shape of the face to which the facial features relate. The information obtained by the 3D sensors may then be used to identify distinctive features on the surface of the face, such as the contour of the eye sockets, nose, and chin. According to some aspects, using facial recognition comprises using machine learning and computer vision based, at least in part, on the distinguishing features and/or the statistical analysis to generate the digital representation of the set of the person's current facial features.

According to some aspects, the method further comprises comparing S11 the digital representation of the set of the person's current facial features to a set of desired facial features. Comparing the digital representation to the set of desired facial features enables determining the differences between the digital representation of the set of the person's current facial features and the digital representation of a set of desired facial features. The comparison can thereby produce input, in particular in the form of the differences between current and desired facial features, for the determination of the substance dispensing plan. The comparison may be performed using an artificial intelligence algorithm configured to determined differences between the digital representation of the set of the person's current facial features and a set of desired facial features. Artificial intelligence algorithms have an advantage in that they may be configured to determine which differences are relevant for the downstream step of determining the substance dispensing plan. Many artificial algorithms, e.g. many machine learning algorithms, can be trained to handle a greater variety of facial features than a parametrized facial feature model is be able provide. A potential application of the disclosed method is to determine a substance dispensing plan for obtaining a more youthful appearance with respect to a current appearance. An important feature relating to old age is wrinkles.

Thus, according to some aspects, the method further comprises determining S12 the presence of one or more wrinkles based on the obtained digital representation of the set of the person's current bodily features.

When the wrinkles have been identified, a substance suitable for smoothing wrinkles can be introduced subcutaneously at a set of regions relating to the wrinkles.

Thus, according to some aspects, the method further comprises determining S22 how much substance to place under the skin of the person to fill the one or more wrinkles based on the comparison between the digital representation of the set of the person's current bodily features and the set of desired bodily features.

When the substance dispensing plan has been determined, it may be executed, e.g. semi-automatically or automatically by a robotic system as described above and below. Thus, according to some aspects, the method comprises dispensing S30 the substance, via a syringe, based on the determined substance dispensing plan.

Thus, according to some aspects, determining the substance dispensing plan comprises determining at least one instruction for control of movement of a robotic arm holding the syringe based on the determined syringe skin penetration location(s) and/or syringe skin penetration attitude(s) and/or syringe skin penetration depth(s).

Further, according to some aspects, determining the substance determining plan comprises determining at least one instruction for dispensing of the substance via the syringe, said at least one instruction for dispensing of the substance of the syringe being correlated to the instructions for control of movement of the robotic arm.

When the substance dispensing plan has been determined, it may be executed, e.g. semi-automatically or automatically by a robotic system as described above and below. Thus, according to some aspects, the method comprises dispensing S30 the substance, via a syringe, based on the determined substance dispensing plan. The present disclosure also relates to a computer program comprising computer program code which, when executed, causes a robotic system to carry out the method as described above and below.

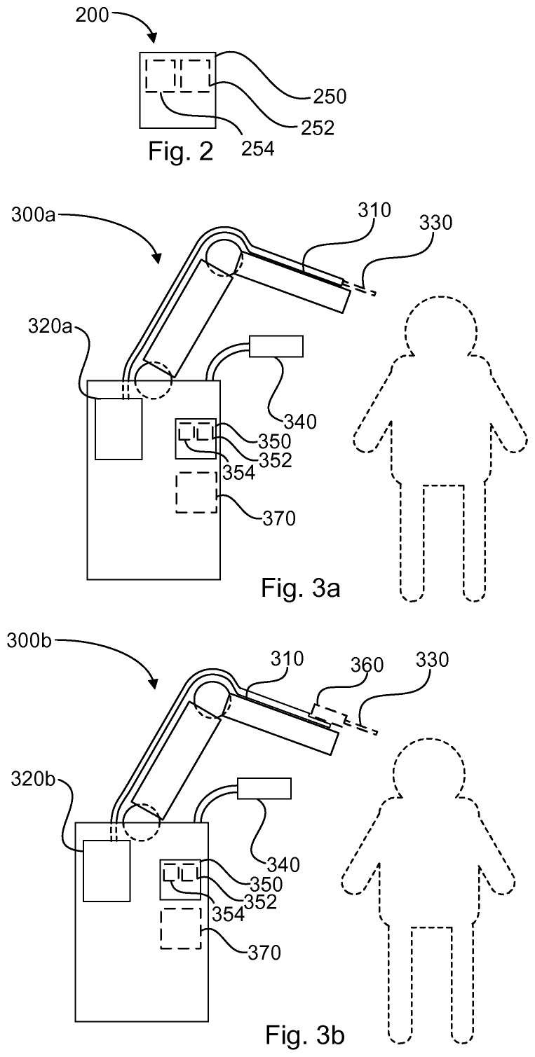

FIG. 2 illustrates a control system 200 for determining a substance dispensing plan. The control system 200 comprises control circuitry 250. The control circuitry 250 is configured to carry out the method as described above and below. According to some aspects, the control circuitry comprises 250 a processor 252 and a memory 254. The memory 254 is configured to store a computer program as described above and below thereon. The processor 252 is configured to execute the computer program stored on the memory. The control system can be integrated into a system having components necessary to carry out the disclosed method, thereby extending the functionality of existing systems or integrating separate systems into a single, larger system with extended functionality.

FIGS. 3 and 3b illustrate robotic systems 300a, 300b for determining and executing a substance dispensing plan. The robotic systems 300a, 300b differ in the manner in which substance may be administered, as will be described further below. The robotic system 300a of

FIG. 3a is configured to administer substance directly, while the robotic system of FIG. 3b is configured to administer substance indirectly, e.g. by acting on a cartridge 360 comprising the substance.

In the following, a robotic system 300a, 300b for determining and executing a substance dispensing plan will be described. The described features apply to the robotic systems 30a, 300b of both FIG. 3a and FIG. 3b, unless stated otherwise.

The robotic system 300a, 300b comprises a robotic arm 310. The robotic system 300a, 300b further comprises a substance delivery system 320a, 320b configured to dispense a substance into the subcutaneous region.

The robotic system 300a, 300b also comprises at least one camera 340. According to some aspects, the robotic system 300a, 300b is configured to move one or more of the at least one camera 340 relative to the person for which the transcutaneous delivery of the substance is intended. The ability to move a camera relative to the person enables scanning a greater area of the person. If the relative motion further comprises the ability to change an attitude of the camera, the person may be scanned from different angles, thereby enabling a three-dimensional scan of the person.

According to some aspects, the at least one camera 340 comprises an infrared-, IR-, camera. According to some further aspects, the robotic system 300a, 300b, e.g. the IR-camera, is configured to emit infrared light. By shining IR light onto the skin of the person for which the transcutaneous delivery of the substance is intended, the IR light may be absorbed by red blood cells inside blood vessels, but scattered back by surrounding tissue. At least some of the IR light scattered back by the surrounding tissue can be detected by the IR-camera, thereby enabling the images captured by the IR-camera to be used as a basis for determining where the person's blood vessels are located. Information enabling the detection of blood vessels may be used to ensure that the robotic system 300a, 300b avoids rupturing blood vessels, e.g. when inserting a syringe. In case the substance is intended to be inserted into the blood stream, e.g. a medicine, the information enabling the detection of blood vessels may be used to ensure that the substance is properly introduced into a blood vessel, e.g. via a syringe. The term infrared light is here to be understood as also comprising near infrared light, i.e. light having a wavelength within a range configured to cause the IR light may be absorbed by red blood cells inside blood vessels, but scattered back by surrounding tissue.

The robotic system 300a, 300b additionally comprises control circuitry 350. The robotic system 300a, 300b may comprise a control system for transcutaneous delivery of substance into a subcutaneous region, as described above and below, wherein the control circuitry 350 of the robotic system 300a, 300b comprises the control circuitry of the control system.

The at least one camera 340 is configured to obtain a digital representation of a set of a person's current bodily features. The at least one camera 340 may further comprise a stereo camera and/or a depth-sensing camera. A stereo camera and/or a depth-sensing camera enable obtaining a three-dimensional digital representation of the set of the person's current bodily features.

The control circuitry 350 is configured to determine a substance dispensing plan based on a comparison between the digital representation of the set of the person's current bodily features and a set of desired bodily features. The robotic system 300a, 300b thereby enables performing automatic methods for determining how to optimally dispense the substance into the subcutaneous region.

The robotic system may however also be configured for manual and/or semi-automatic use based on the determined substance dispensing plan.

According to some aspects, the robotic system 300a, 300b comprises and interface 370 configured to provide the determined substance dispensing plan, e.g. via a display, a cable interface, such as a universal serial bus, USB, interface and/or a wireless interface, such as a WiFi-interface. The interface 370 may further be configured to receive command signals from a user, e.g. via a keyboard, a touch screen, a cable interface, such as a universal serial bus, USB, interface and/or a wireless interface, such as a WiFi-interface. The interface 370 thereby enables manual and/or semi-automatic control of the robotic system 300a, 300b, e.g. to carry out the determined substance dispensing plan. The interface 370 further enables a user to examine the determined substance dispensing plan before it is performed.

The robotic system 300a, 300b further enables the integration of artificial intelligence methods for determining the substance dispensing plan, which may provide a more accurate substance dispensing plan than a human counterpart would be able to do. In other words, the robotic system 300a, 300b is thereby able to provide plans for dispensing a substance into a subcutaneous region faster and more accurate than a human counterpart would be able to do. The improved accuracy of the substance dispensing plan translates to reduced treatment times and/or a reduced probability for complications, while simultaneously achieving superior results compared to the technology of the prior art.

Thus, according to some aspects, the substance delivery system 320a, 320b is configured to dispense the substance based on the determined substance dispensing plan.

According to some aspects, the control circuitry 350 comprises a processor 352 and a memory 354. The memory 354 is configured to store a computer program for transcutaneous delivery of a substance into a subcutaneous region, as described above and below, thereon. The processor 352 is configured to execute the computer program stored on the memory 354.

The substance delivery system 320a, 320b may be configured to dispense the substance either directly or indirectly. For instance, according to some aspects the substance delivery system 320a comprises a syringe 330 arranged at the robotic arm 310. The substance delivery system 320a is configured to dispense the substance via the syringe 330. The substance delivery system 320a is thereby configured to dispense the substance directly via the syringe 330. According to some aspects, the substance delivery system 320b is configured to receive a cartridge 360 comprising a syringe 330. The cartridge 360 comprises the substance. The substance delivery system 320b is configured to dispense the substance via the syringe 330. The substance delivery system 320b is thereby configured to dispense the substance indirectly by acting on an external object in the form of a cartridge 360 comprising a syringe 330.

According to some aspects, the substance dispensing plan comprises at least one of a syringe skin penetration location, a syringe skin penetration attitude, and a syringe skin penetration depth.

According to some aspects, the system is adapted to determine the substance dispensing plan by determining at least one instruction for control of movement of a robotic arm holding the syringe based on the determined syringe skin penetration location(s) and/or syringe skin penetration attitude(s) and/or syringe skin penetration depth(s).

Further, according to some aspects, the system is further adapted to determine the substance determining plan by determining at least one instruction for dispensing of the substance via the syringe, said at least one instruction for dispensing of the substance of the syringe being correlated to the instructions for control of movement of the robotic arm.

According to some aspects, the substance dispensing plan comprises dispensing substance at a substance dispensing volume at a subcutaneous region of the person. According to some aspects, the substance dispensing plan comprises dispensing anaesthesia, e.g. at the subcutaneous region.

These are factors that an artificial intelligence algorithm is particularly suitable for determining. Since artificial intelligence algorithms, e.g. in the field of machine learning, herein considered to be a subfield of artificial intelligence, may be trained to perform many tasks at or above human-level performance, the disclosed robotic system 320a, 320b may be automatized to operate in a semi-automatic or automatic manner with results at or above human-level performance. Not only may procedures, e.g. in the field of cosmetic surgery, result in a more aesthetically pleasing outcome, but may also save time and/or reduce the risk of complications when implemented according to the determined substance dispensing plan. Thus, according to some aspects, the control circuitry 350 is configured to determine the substance dispensing plan using, at least in part, an artificial intelligence algorithm for making the comparison between the digital representation of the set of the person's current bodily features and the set of desired bodily features.

In order to be able to effectively provide wide range of possible syringe skin penetration locations, syringe skin penetration attitudes, and syringe skin penetration depths, the robotic arm may be configured to move in six degrees of freedom. The ability to move in six degrees of freedom greatly extends the range of possible treatments as well as the degree to which a desired result can be achieved. In particular, a six degree of freedom robotic arm is able to perform a transcutaneous penetration at a wide range of attitudes.

According to some aspects, the control circuitry is further configured to compare the digital representation of the set of the person's current bodily features to a digital representation of a set of desired bodily features. Comparing the digital representation to the set of desired bodily features enables determining the differences between the digital representation of the set of the person's current bodily features and the digital representation of a set of desired bodily features. The comparison helps identifying regions on the surface of the person differing sufficiently from the set of desired bodily features. The comparison can thereby produce input, in particular in the form of the differences between current and desired bodily features, for the determination of the substance dispensing plan. A great advantage of the comparison is that, in combination with an artificial intelligence algorithm, the act of comparing may be used to train the artificial intelligence algorithm to identify criteria for determining the substance dispensing plan.

According to some aspects, the at least one camera is configured to scan the person's face. The control circuitry is configured to generate a digital representation of a set of the person's current facial features based on the scan. Scanning the person's face is a quick and accurate way to obtain a digital representation of a set of the person's current facial features. Scanning the person's face further enables obtaining a digital representation from different angles and at different distances, thereby enabling obtaining an accurate, three-dimensional digital representation of the set of the person's current facial features.

According to some aspects, the control circuitry is configured to generate the digital representation of the set of the person's current facial features, at least in part, using facial recognition. Facial recognition algorithms enable identifying distinguishing features and/or performing statistical analysis of the scan of the person's face in order to distill the scan into values and comparing the values with templates to eliminate variances. The control circuitry may be configured to use facial recognition in combination with 3D sensors in order to capture information about the shape of the face to which the facial features relate. The information obtained by the 3D sensors may then be used to identify distinctive features on the surface of the face, such as the contour of the eye sockets, nose, and chin. According to some aspects, the control circuitry is configured to use machine learning and computer vision based, at least in part, on the distinguishing features and/or the statistical analysis to generate the digital representation of the set of the person's current facial features when using facial recognition.

According to some aspects, the comparison between the digital representation of the set of the person's current bodily features and the set of desired bodily features comprises a comparison between the digital representation of the set of the person's current facial features and a set of desired facial features. Comparing the digital representation to the set of desired facial features enables determining the differences between the digital representation of the set of the person's current facial features and the digital representation of a set of desired facial features. The comparison can thereby produce input, in particular in the form of the differences between current and desired facial features, for the determination of the substance dispensing plan.

According to some aspects, the control circuitry 350 is further configured to determine the presence of one or more wrinkles based on the obtained digital representation of the set of the person's current bodily features. According to some further aspects, the control circuitry 350 is configured to determine how much substance to place under the skin of the person to fill the one or more wrinkles based on the comparison between the digital representation of the set of the person's current bodily features and the set of desired bodily features. The robotic system 320a, 320b is thereby configured to identify facial features related to the appearance of an old person, and to carry out the necessary steps in providing a more youthful appearance to the person. In order to effectively smooth the one or more wrinkles, the substance delivery system 320a, 320b may be configured to dispense hyaluronic acid via the syringe 330.

The functionality of the robotic system 320a, 320b may be further extended by adding the ability to remove blood and/or tissue and including said functionality as part of the substance dispensing plan. For instance, before or instead of, smoothing out one or more wrinkles, it may be desirable to reduce subcutaneous fat content. Thus, according to some aspects, the robotic system 300a, 300b is further configured to suck out fat from the subcutaneous region. The robotic system 300a, 300b may comprise a liposuction system comprising a suction syringe and a suction mechanism configured to such fat from the subcutaneous region via the suction syringe. The substance dispensing plan may further comprise a step of sucking out fat from the subcutaneous region. The robotic system 300a, 300b is thereby configured for liposuction in combination with dispensing the substance. According to some aspects, the step of dispensing a substance is omitted while the substance dispensing plan comprises steps comprising liposuction. The robotic system 300a, 300b is thereby configured for liposuction in a manual, semi-automatic and/or automatic manner.

To sum up, the disclosed robotic systems are configured to implement the methods disclosed in relation to FIG. 1 and may comprise control systems as disclosed in relation to FIG. 2. Thus, all the technical features disclosed in relation to FIGS. 1 and 2 may be included, mutatis mutandis, into the disclosed robotic systems, and vice versa.

The present invention is not limited to the above-described preferred embodiments. Various alternatives, modifications and equivalents may be used. Therefore, the above embodiments should not be taken as limiting the scope of the invention, which is defined by the appending claims.

1. A computer implemented method for determining a substance dispensing plan, the method comprising
obtaining a digital representation of a set of a person's current bodily features, and
determining a substance dispensing plan based on a comparison between the digital representation of the set of the person's current bodily features and a set of desired bodily features.

2. The method according to claim 1, further comprising
comparing the digital representation of the set of the person's current bodily features to a digital representation of a set of desired bodily features.

3. The method according to, claim 1, wherein obtaining a digital representation of a set of a person's current bodily features further comprises:
scanning the person's face, and
generating a digital representation of a set of the person's current facial features.

4. The method according to claim 3, wherein the step of generating a digital representation of a set of the person's current facial features is performed, at least in part, using facial recognition.

5. The method according to claim 3, further comprising
comparing the digital representation of the set of the person's current facial features to a set of desired facial features.

6. The method according to claim 5, further comprising
determining how much substance to place under the skin of the person to fill the one or more wrinkles based on the comparison between the digital representation of the set of the person's current bodily features and the set of desired bodily features.

7. The method according to claim 1, wherein determining the substance dispensing plan comprises
using, at least in part, an artificial intelligence algorithm for making the comparison between the digital representation of the set of the person's current bodily features and the set of desired bodily features.

8. The method according to claim 1, wherein the substance dispensing plan comprises at least one of a
syringe skin penetration location(s),
syringe skin penetration attitude(s), or
syringe skin penetration depth(s).

9. The method according to claim 8, wherein determining the substance dispensing plan comprises determining at least one instruction for control of movement of a robotic arm holding the syringe based on the determined syringe skin penetration location(s) and/or syringe skin penetration attitude(s) and/or syringe skin penetration depth(s).

10. The method according to claim 9, wherein determining the substance dispensing plan comprises determining at least one instruction for dispensing of the substance via the syringe, said at least one instruction for dispensing of the substance of the syringe being correlated to the instructions for control of movement of the robotic arm.

11. The method according to claim 1, further comprising
dispensing the substance, via a syringe, based on the determined substance dispensing plan.

12. The method according to claim 1, wherein the substance dispensing plan comprises dispensing substance at a substance dispensing volume at a subcutaneous region of the person.

13. The method according to claim 1, further comprising
determining the presence of one or more wrinkles based on the obtained digital representation of the set of the person's current bodily features.

14. A computer program comprising computer program code which, when executed, causes a robotic system to carry out the method according to claim 1.

15. A control system for determining a substance dispensing plan, the control system comprising
control circuitry,
wherein the control circuitry is configured to carry out the method according to claim 1.

16. A control system for determining a substance dispensing plan, the control system comprising a control circuitry, wherein the control circuitry comprises
a processor; and
a memory,
wherein the memory is configured to store a computer program comprising computer program code which, when executed, causes a robotic system to carry out the method of claim 1, and
wherein the processor is configured to execute the computer program stored on the memory.

17. A robotic system for determining and executing a substance dispensing plan, the robotic system comprising:
a robotic arm,
a substance delivery system configured to dispense the substance into a subcutaneous region,
at least one camera,
control circuitry,
wherein the at least one camera is configured to obtain a digital representation of a set of a person's current bodily features, wherein the control circuitry is configured to determine a substance dispensing plan based on a comparison between the digital representation of the set of the person's current bodily features and a set of desired bodily features.

18. The robotic system according to claim 17, wherein the substance delivery system is configured to dispense the substance based on the determined substance dispensing plan.

19. The robotic system according to claim 17, wherein the control circuitry comprises a processor and a memory, wherein the memory is configured to store a computer program comprising computer program code which, when executed, causes the robotic system to dispense the substance at a substance dispensing volume into the subcutaneous region of the person and wherein the processor is configured to execute the computer program stored on the memory.

20. The robotic system according to claim 17, wherein the substance delivery system comprises a syringe arranged at the robotic arm, and
wherein the substance delivery system is configured to dispense the substance via the syringe.

21. The robotic system according to claim 17, wherein the substance delivery system is configured to receive a cartridge comprising a syringe, the cartridge comprising the substance, and wherein the substance delivery system is configured to dispense the substance via the syringe.

22. The robotic system according to claim 17, wherein the robotic arm is configured to move in six degrees of freedom.

23. The robotic system according to claim 17, wherein the control circuitry is further configured to compare the digital representation of the set of the person's current bodily features to a digital representation of a set of desired bodily features.

24. The robotic system according to claim 17, wherein the at least one camera is configured to scan the person's face, and wherein the control circuitry is configured to generate a digital representation of a set of the person's current facial features based on the scan.

25. The robotic system according to claim 24, wherein the control circuitry is configured to generate the digital representation of the set of the person's current facial features, at least in part, using facial recognition.

26. The robotic system according to claim 24, wherein the comparison between the digital representation of the set of the person's current bodily features and the set of desired bodily features comprises a comparison between the digital representation of the set of the person's current facial features and a set of desired facial features.

27. The robotic system according to claim 17, wherein the control circuitry is configured to determine the substance dispensing plan using, at least in part, an artificial intelligence algorithm for making the comparison between the digital representation of the set of the person's current bodily features and the set of desired bodily features.

28. The robotic system according to claim 17, wherein the substance dispensing plan comprises at least one of a
syringe skin penetration location,
syringe skin penetration attitude, or
syringe skin penetration depth.

29. The robotic system according to claim 28, wherein the system is adapted to determine the substance dispensing plan by determining at least one instruction for control of movement of a robotic arm holding the syringe based on the determined syringe skin penetration location(s) and/or syringe skin penetration attitude(s) and/or syringe skin penetration depth(s).

30. The robotic system according to claim 29, wherein the system is adapted to determine the substance dispensing plan by determining at least one instruction for dispensing of the substance via the syringe, said at least one instruction for dispensing of the substance of the syringe being correlated to the instructions for control of movement of the robotic arm.

31. The robotic system according to claim 29, wherein the system is adapted to execute the determined at least one instruction for control of movement of a robotic arm and/or at least one instruction for dispensing of the substance via the syringe.

32. The robotic system according to claim 17, wherein the substance dispensing plan comprises dispensing substance at a substance dispensing volume at a subcutaneous region of the person.

33. The robotic system according to claim 17, wherein the control circuitry is further configured to determine the presence of one or more wrinkles based on the obtained digital representation of the set of the person's current bodily features.

34. The robotic system according to claim 33, wherein the control circuitry is further configured to determine how much substance to place under the skin of the person to fill the one or more wrinkles based on the comparison between the digital representation of the set of the person's current bodily features and the set of desired bodily features.

35. The robotic system according to claim 17, wherein the substance delivery system is configured to dispense hyaluronic acid via a syringe.
